(19) United States
(12) Patent Application Publication
(10) Pub. No.: US 2025/0314649 A1
(43) Pub. Date: Oct. 9, 2025

(54) RAMAN-ACTIVE NANOPARTICLE FOR SURFACE-ENHANCED RAMAN SPECTROSCOPY AND METHOD OF PRODUCING THE SAME

(71) Applicant: KOREA RESEARCH INSTITUTE OF STANDARDS AND SCIENCE, Daejeon (KR)

(72) Inventors: Eun-Ah YOU, Daejeon (KR); Ryeong Myeong KIM, Daejeon (KR)

(21) Appl. No.: 18/802,356

(22) Filed: Aug. 13, 2024

(30) Foreign Application Priority Data
Apr. 8, 2024 (KR) 10-2024-0047559

(51) Int. Cl. G01N 33/543; G01N 21/65; G01N 33/531

(52) U.S. Cl. CPC G01N 33/54346; G01N 21/658; G01N 33/531

(57) ABSTRACT

A Raman-active nanoparticle of the present disclosure includes a spherical plasmonic metal core; a plasmonic metal shell having surface irregularities; and a first self-assembled monolayer that binds to each of the core and the shell, is positioned between the core and the shell, and includes a Raman reporter satisfying the following Chemical Formula 1:

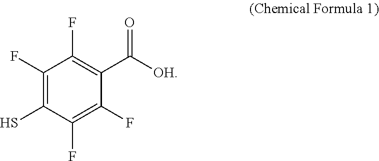 [SMILES: O=C(O)c1c(F)c(F)c(S)c(F)c1F]

### CROSS-REFERENCE TO RELATED APPLICATION

This application claims priority to Korean Patent Application No. 10-2024-0047559, filed on Apr. 8, 2024, the disclosure of which is hereby incorporated by reference in its entirety.

### TECHNICAL FIELD

The following disclosure relates to a Raman-active nanoparticle for surface-enhanced Raman spectroscopy having excellent biocompatibility in which a self-assembled monolayer having high chemical stability is introduced, and a method of producing the same.

### BACKGROUND

Surface-enhanced Raman spectroscopy (SERS) is Raman spectroscopy utilizing a phenomenon in which a Raman scattering signal of molecules adsorbed on a microstructure of a metal surface is enhanced, and is spectroscopy utilizing a phenomenon in which Raman scattering intensity rapidly increases by 10⁶ to 10⁸ times or more when molecules are adsorbed on a surface of a metal nanostructure. The Raman spectroscopy directly provides information about an oscillation state of molecules or a molecular structure, and is recognized as a powerful analysis method for ultra-sensitive chemical, biological, and biochemical analysis.

The SERS fused with nanotechnology, which is currently developing at a very rapid pace, is particularly greatly expected to be efficiently used as a medical sensor. As an example, currently, studies for carrying out the initial diagnosis of various diseases comprising Alzheimer's disease or diabetes together with high-sensitivity DNA analysis using the SERS have been actively conducted.

The present inventors have improved the reproducibility of measurement by developing a Raman-active particle having a core-shell structure using a previously known Raman reporter, as disclosed in Korean Patent Laid-Open Publication No. 10-2365091.

However, a Raman reporter containing a nitro group (—NO₂) may have a toxic effect on the central nervous system and may cause harm to the blood, liver, kidneys, and the like when exposed chronically, making it difficult to use in the biofield. In addition, as the chemical stability of the molecular layer is reduced, there is a risk of side reactions easily occurring.

Therefore, there is a need to develop a Raman-active particle that has excellent biocompatibility, is chemically stable, has a high sensitivity capable of detection at a single molecule level, and may implement detection with improved reliability and reproducibility.

### SUMMARY

An embodiment of the present disclosure is directed to providing a Raman-active nanoparticle that has strictly defined hot spots, exhibits uniform Raman activity per nanoparticle, and at the same time, uniform Raman activity between particles, and may implement detection with reproducibility and reliability.

Another embodiment of the present disclosure is directed to providing a Raman-active nanoparticle that may implement detection at a single molecule level and has extremely excellent uniformity and sensitivity.

Still another embodiment of the present disclosure is directed to providing a Raman-active nanoparticle that has excellent biocompatibility and is suitable for detecting a specific biomarker or a cell surface receptor.

Still another embodiment of the present disclosure is directed to providing a Raman-active nanoparticle comprising a self-assembled monolayer having improved chemical stability.

Still another embodiment of the present disclosure is directed to providing a method of producing Raman-active nanoparticles that have excellent shape reproducibility when produced under the same conditions, implement detection with reproducibility and reliability, and have extremely excellent sensitivity.

Still another embodiment of the present disclosure is directed to providing a method of producing Raman-active nanoparticles that may be mass-produced at room temperature in a short time by a simple method and have excellent commerciality.

In one general aspect, a Raman-active nanoparticle comprises a spherical plasmonic metal core; a plasmonic metal shell having surface irregularities; and a first self-assembled monolayer that binds to each of the core and the shell, is positioned between the core and the shell, and comprises a Raman reporter satisfying the following Chemical Formula 1:

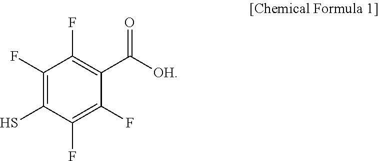 [SMILES: O=C(O)c1c(F)c(F)c(S)c(F)c1F]

In the Raman-active nanoparticle, the surface of the shell may further comprise a second self-assembled monolayer comprising a Raman reporter satisfying Chemical Formula 1.

In the Raman-active nanoparticle, the plasmonic metal core and the plasmonic metal shell may be independently one or more metals selected from gold, silver, platinum, palladium, nickel, aluminum, and copper.

The plasmonic metal core and the plasmonic metal shell may be the same metal.

In another general aspect, a method of producing Raman-active nanoparticles comprises: a) forming a first self-assembled monolayer comprising a Raman reporter satisfying the following Chemical Formula 1 on a spherical plasmonic metal core; and b) forming a plasmonic metal shell that surrounds the metal core on which the self-assembled monolayer is formed and has surface irregularities using a reaction solution in which a buffer solution, the metal core on which the self-assembled monolayer is formed, and a plasmonic metal precursor are mixed:

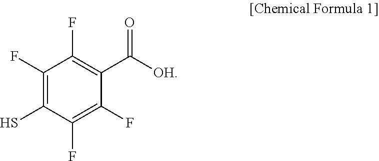 [SMILES: O=C(O)c1c(F)c(F)c(S)c(F)c1F]

The method of producing Raman-active nanoparticles may further comprise, after step b), c) forming a second self-assembled monolayer comprising a Raman reporter satisfying Chemical Formula 1 on the plasmonic metal shell.

Other features and aspects will be apparent from the following detailed description, the drawings, and the claims.

### BRIEF DESCRIPTION OF THE DRAWINGS

FIG. 1 is a diagram showing a scanning electron microscope (SEM) image obtained by observing Raman-active nanoparticles produced according to an exemplary embodiment.

FIGS. 2A and 2B are diagrams showing transmission electron microscope (TEM) images obtained by observing Raman-active nanoparticles produced according to an exemplary embodiment.

FIG. 3 is a diagram showing a spectrum of light emitted from a subject by excitation light using an exemplary embodiment.

FIG. 4 is a diagram showing the result of evaluating Raman signal intensity using a slope value obtained by linear regression analysis for all concentration values using an exemplary embodiment.

### DETAILED DESCRIPTION OF EMBODIMENTS

Hereinafter, a Raman-active nanoparticle of the present disclosure and a method of producing the same will be described in detail with reference to the accompanying drawings. The drawings to be provided below are provided by way of example so that the spirit of the present disclosure can be sufficiently transferred to those skilled in the art. Therefore, the present disclosure is not limited to the drawings to be provided below, but may be modified in many different forms. In addition, the drawings provided below may be exaggerated in order to clarify the spirit of the present disclosure. Technical terms and scientific terms used herein have the general meanings understood by those skilled in the art to which the present disclosure pertains unless otherwise defined, and a description for the known function and configuration unnecessarily obscuring the gist of the present disclosure will be omitted in the following description and the accompanying drawings. In addition, unless the context clearly indicates otherwise, singular forms used in the specification and the scope of the appended claims are intended to comprise plural forms. A unit used in the present specification and the appended claims without special mention is based on weight, and as an example, a unit of % or a ratio refers to wt % or a weight ratio.

A Raman-active nanoparticle according to an aspect of the present disclosure comprises a spherical plasmonic metal core; a plasmonic metal shell having surface irregularities; and a first self-assembled monolayer that binds to each of the core and the shell, is positioned between the core and the shell, and comprises a Raman reporter satisfying the following Chemical Formula 1:

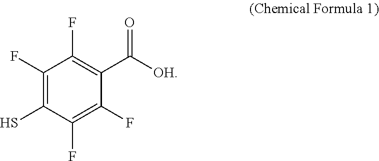 [SMILES: O=C(O)c1c(F)c(F)c(S)c(F)c1F]

Specifically, surface plasmon enhancement in a metal nanostructure may be highly limited to a specific position, which is called localized surface plasmon resonance (LSPR), and the area is called a hot spot area. In particular, when a Raman-active molecule is positioned at a hot spot of a plasmonic nanostructure, a surface-enhanced Raman scattering (hereinafter, referred to as SERS) effect is obtained, and the hot spot is highly limited to a spatially narrow area like a nanogap, which is called a SERS hot spot.

The Raman-active nanoparticle of the present disclosure has a core-shell structure, and comprises a self-assembled monolayer that is positioned between the core and the shell and on a surface of the shell and comprises a Raman reporter represented by a chemical formula having a sulfhydryl group (—HS), which is a surface binding functional group, in a coumarin parent, and a methyl group bonded to carbon at the 4-position, and therefore, a nanogap corresponding to a thickness of a self-assembled monolayer having a strictly controlled thickness due to the characteristic of self-assembly may be formed in the Raman-active nanoparticle of the present disclosure. Since the Raman-active nanoparticle of the present disclosure contains a relatively environmentally friendly compound, the Raman-active nanoparticle of the present disclosure may be suitable for detecting a specific biomarker or a cell surface receptor due to excellent biocompatibility, and may have improved detection reliability due to chemical stability.

In addition, since the shape of the plasmonic metal core is spherical, the self-assembled monolayer has a spherical shape, and in the plasmonic metal shell, the inner shape of the metal shell in contact with the first self-assembled monolayer may also have a spherical shape. Accordingly, the nanogap may be positioned in the entire area of the Raman-active nanoparticle, and the nanogap having a uniform size may also be positioned in all directions based on a radical direction.

In particular, since the first self-assembled monolayer positioned between the core and the shell comprises the Raman reporter satisfying Chemical Formula 1, the Raman reporter is positioned at positions that are well-defined and radically identical in the Raman-active nanoparticle, and the Raman reporter that is uniformly positioned at a high density in the entire area of the Raman-active nanoparticle is positioned at the nanogap, that is, the hot spot where surface plasmon resonance occurs locally. In other words, in the Raman-active nanoparticle of the present disclosure, the Raman reporter satisfying Chemical Formula 1 is positioned at the hot spot, such that a SERS effect is obtained. As a result, the Raman-active nanoparticle may have a SERS hot spot area that is uniformly present in the entire area of the Raman-active nanoparticle.

In addition, in the Raman-active nanoparticle of the present disclosure, the surface of the shell may further comprise a second self-assembled monolayer comprising a Raman reporter satisfying Chemical Formula 1.

In the present disclosure, an additional process of forming a second self-assembled monolayer comprising a Raman reporter satisfying Chemical Formula 1 on the surface of the metal shell is further comprised, such that sensitivity may be further improved compared to previously reported Raman-active nanoparticles.

Furthermore, regarding a method of producing Raman-active nanoparticles described below, the Raman reporter satisfying Chemical Formula 1 allows irregularities having a uniform size to be formed in the entire area of the surface of the metal shell, such that isotropic Raman activity may be obtained in the nanoparticles, these Raman-active nanoparticles may exhibit uniform SERS activity based on the particles, uniform SERS activity between the particles may be exhibited because there is almost no deviation in Raman activity between the particles, and furthermore, as the surface of the metal shell has an irregular structure, a strong electromagnetic field is formed to significantly improve Raman intensity; thus, Raman signal intensity may be excellent and a high correlation may be exhibited as compared to the particles according to the related art.

Here, the correlation may refer to a relation with the number of Raman-active nanoparticles in which a SERS signal is detected, among all Raman-active nanoparticles in 2D mapping. That is, when a SERS signal is detected in a large amount of Raman-active nanoparticles based on the total number of Raman-active nanoparticles, the correlation may be said to be high.

In addition, in the Raman-active nanoparticle according to an exemplary embodiment of the present disclosure, well-defined hot spots are continuously present in the entire area of the nanoparticle, and the Raman reporter satisfying Chemical Formula 1 is uniformly positioned in the well-defined hot spots, such that a biochemical material (biomaterial) having a several to several tens of micrometers may also be reproducibly detected.

As a specific example, the plasmonic metal shell may comprise plasmonic metal fine particles having an average size of 0.1 D to 2 D based on a diameter (D) of the metal core, and may have surface irregularities due to the plasmonic metal fine particles.

Specifically, the metal shell in a state of binding to the first self-assembled monolayer may be formed of metal fine particles having an average size of 0.1 D to 2 D, specifically 0.3 D to 1 D, more specifically 0.5 D to 1 D, and still more specifically 0.5 D to 0.8 D, based on the diameter (D) of the metal core, and the metal shell may have irregularities having a uniform size according to the particle shape of the metal fine particle. The metal fine particles forming the metal shell have the average size in the range described above based on the diameter of the metal core, such that the Raman signal intensity may be improved compared to the intensity according to the related art.

Specifically, since the irregular structure due to the metal fine particles of the plasmonic metal shell may have a uniform size by the Raman reporter satisfying Chemical Formula 1 and the metal shell formed of the metal fine particles having the average size in the range described above, as described above, hot spots on the surface of the shell itself together with hot spots by the nanogap between the metal core and the metal shell may be formed, that is, hot spots may be formed according to a spaced distance between the closest irregularities having a uniform size, which is more advantageous for Raman signal enhancement.

As an exemplary embodiment, the size of the Raman-active nanoparticle having a core-shell structure may be 80 to 200 nm, specifically 100 to 150 nm, and more specifically 110 to 140 nm.

As a specific example, a thickness of the shell in the core-shell structure may be 15 to 60 nm, preferably 20 to 50 nm, and more preferably 25 to 40 nm. In this case, the thickness of the shell may refer to a distance from the surface of the core to the outmost part of the shell.

The metal fine particles themselves in the metal shell of the Raman-active nanoparticle of the present disclosure protrude to form bumpy irregularities in the entire area of the surface of the metal shell, such that the sensitivity of the Raman-active nanoparticle may be increased by the metal shell, uniform Raman activity may be exhibited in one particle, and uniformity of Raman activity between the particles may not be inhibited.

An average diameter of the plasmonic metal core may be 20 to 100 nm, specifically 30 to 80 nm, and more specifically 40 to 60 nm.

When the average diameter of the plasmonic metal core is 20 nm or more or 30 nm or more, a radius of curvature is appropriate for forming the first self-assembled monolayer comprising the Raman reporter represented by Chemical Formula 1, and a nanogap having a uniform size may be present by the first self-assembled monolayer by interaction between the metal core and the Raman reporter; thus, it is preferable that the average diameter of the plasmonic metal core satisfies the above range. However, when the average diameter of the plasmonic metal core is less than 20 nm, a curvature is excessively large and a radius of curvature is small, which makes it difficult for the Raman reporter to form a dense first self-assembled monolayer on the surface of the core. As a result, it is difficult to effectively form irregularities in the entire area of the metal shell, and the uniformity of Raman activity is significantly reduced, which is not preferable.

In a specific example, the self-assembled monolayer may be the self-assembled monolayer of the Raman reporter, and the Raman reporter may refer to an organic compound (organic molecule) comprising a Raman-active molecule or an organic compound (organic molecule) having a binding force to the metal of the metal core and comprising a Raman-active molecule.

The Raman reporter has a binding force to the metal of the metal core, such that the first self-assembled monolayer of the Raman reporter may be formed on the metal core to which a pure metal surface is exposed.

The Raman-active molecule may comprise a surface-enhanced Raman-active molecule, a surface-enhanced resonance Raman-active molecule, a hyper Raman-active molecule, or a coherent Van stokes Raman-active molecule, and the Raman-active molecule may have a Raman signal, and may also have both a Raman signal and a fluorescence signal.

Specifically, in the related art, the nanogap is positioned in the entire area of the Raman-active particle, but since the size of the nanogap varies depending on the position, the enhancement of the Raman signal intensity is limited, and reproducibility of detection of a target material is reduced. On the other hand, in the Raman-active nanoparticle of the present disclosure, the self-assembled monolayer comprising the Raman reporter satisfying Chemical Formula 1 may have a spherical shape that is significantly similar to the radius of curvature of the spherical metal core, and thus, the size of the nanogap positioned in the entire area of the Raman-active nanoparticle is uniform, such that Raman signal intensity may be improved.

In particular, when the average diameter of the plasmonic metal core is in the range described above, the self-assembled monolayer formed by the interaction with the Raman reporter satisfying Chemical Formula 1 forms a SERS hot spot area that is uniformly present in the entire area of the Raman-active nanoparticle, such that the Raman signal intensity may be significantly improved.

In addition, as the nanogap is formed between the metal core and the metal shell by the Raman reporter bound to the metal core, it is preferable that a length (size) of the Raman reporter is 2.5 nm or less, specifically 0.5 to 2.0 nm, and more specifically 0.8 to 1.2 nm, in terms of having stronger sensitivity. In this case, the length (size) of the Raman reporter also corresponds to the thickness of the self-assembled monolayer.

In a specific example, each of the plasmonic metal core and the plasmonic metal shell may be a metal generating surface plasmon by an interaction with light. As an example, each of the plasmonic metal core and the plasmonic metal shell may be gold, silver, platinum, palladium, nickel, aluminum, copper, a mixture thereof, an alloy thereof, or the like. However, each of the plasmonic metal core and the plasmonic metal shell may be gold or silver, considering biocompatibility.

As another specific example, the plasmonic metal core and the plasmonic metal shell may be the same metal, and as an example, the plasmonic metal core and the plasmonic metal shell may be gold.

In a specific example, the Raman-active nanoparticle may further comprise a receptor that is fixed to the plasmonic metal shell and binds to an analyte. The receptor may be any material known to specifically bind to an analyte, such as complementary binding between enzyme-substrate, antigen-antibody, protein-protein, or DNAs. In this case, the receptor may comprise a functional group that spontaneously binds to the metal of the metal shell (as an example, a thiol group, a carboxyl group, an amine group, or the like), and may be in the state of being spontaneously and chemically bound to the metal shell by the functional group.

In a specific example, the Raman-active nanoparticle may further comprise a blocking molecule that covers a surface area of the shell to which the receptor is not attached (bound). The blocking molecule prevents an undesired interaction between the shell surface itself, not the receptor, and the analyte, and may serve to make orientation of the receptor positioned on the surface of the shell more constant. The blocking molecule may be any material that is commonly used for preventing nonspecific binding on the metal surface in the biosensor field, such as bovine serum albumin (BSA).

The analyte may be a material derived from a living organism (comprising a virus) or a non-living organism. The material derived from a living organism may comprise a cell component. Specifically, the analyte may comprise a lesion biomarker having lesion specificity, a pathogen, a protein, a nucleic acid, a sugar, a drug, and the like. More specifically, the analyte may be an amino acid, a peptide, a polypeptide, a protein, a glycoprotein, a lipoprotein, a nucleoside, a nucleotide, an oligonucleotide, a nucleic acid, a sugar, a carbohydrate, an oligosaccharide, a polysaccharide, a fatty acid, a lipid, a hormone, a metabolite, a cytokine, a chemokine, a receptor, a neurotransmitter, an antigen, an allergen, an antibody, a substrate, a cofactor, an inhibitor, a drug, a pharmaceutical material, a nutritional substance, a prion, a toxin, a poisonous material, an explosive material, an insecticide, a chemical weapon agent, a biohazard agent, a radioactive isotope, a vitamin, a heterocyclic aromatic compound, a carcinogen, a mutagen, an anesthetic, an amphetamine, a barbiturate, a hallucinogen, a waste, or a pollutant. In addition, when the analyte is a nucleic acid, the nucleic acid may comprise genes, viral RNA and DNA, bacterial DNA, fungal DNA, mammalian DNA, cDNA, mRNA, RNA, and DNA fragments, oligonucleotides, synthetic oligonucleotides, modified oligonucleotides, single-stranded and double-stranded nucleic acids, natural and synthetic nucleic acids, and the like.

The analyte may be positioned in-vivo, and may be detected in-vivo. That is, the Raman-active nanoparticle described above may be for use in-vivo, and for biological injection.

On the contrary, the analyte may be positioned in-vitro, and may be detected in-vitro. That is, the Raman-active nanoparticle described above may be for use in-vitro. In this case, the analyte may be in the form of a sample collected in-vivo such as blood, urine, mucosal detachment, saliva, body fluids, tissues, biopsies, a combination thereof, or the like, but is not limited thereto.

In a specific example, the Raman-active nanoparticle may be used for near-infrared excitation light having a wavelength of, specifically 700 nm to 850 nm, more specifically 750 nm to 800 nm, and still more specifically 780 nm to 790 nm. That is, the Raman-active nanoparticle may implement detection and analysis of the analyte by light irradiation in a near-infrared region.

In a specific example, the Raman-active nanoparticle may have a strong Raman signal at 1,035 to 1,075 cm⁻¹, 1,150 to 1,190 cm⁻¹, and 1,570 to 1,610 cm⁻¹ when irradiated with a 785 nm light source.

As is known, when a biomaterial comprising a biochemical material is irradiated with visible light, a fluorescence phenomenon may occur. Since fluorescence intensity is significantly strong compared to Raman scattering and fluorescence occurs in a region similar to that of Raman scattering, it is difficult to obtain a pure Raman spectrum because the Raman spectrum overlaps with a fluorescence peak. Therefore, SERS analysis through irradiation with light having a near-infrared band rather than visible light is significantly advantageous in the biofield because the Raman spectrum may be obtained without influence of fluorescence.

Substantially, when an analyte is detected using the Raman-active nanoparticles according to a specific example, basal fluorescence may not appear substantially on a Raman spectrum of the analyte obtained by near-infrared irradiation.

In an exemplary embodiment, a SERS signal in the Raman mapping may be detected in 80% or more, specifically 85% or more, more specifically 90% or more, and unlimitedly 95% or less of the total number of Raman-active nanoparticles. As the SERS signal in the Raman mapping is detected in the Raman-active nanoparticles in the above range, detection reliability and reproducibility of a target material may be significantly improved, which is preferable.

In this case, the Raman mapping may be Raman mapping for an area having a predetermined size, and the predetermined size may be (1 to 100 μm)×(1 to 100 μm), but is not limited thereto. In addition, a mapping interval in the Raman mapping may be at a level of 0.1 μm to 10 μm for each of the axes perpendicular to each other, an output of excitation light (excitation laser light) may be at a level of 1 mW to 90 mW, and as a practical example, at a level of 1 mW to 10 mW, an excitation light irradiation time may be 0.5 to 10 seconds, and the number of times of scanning may be 1 to 5, but the present disclosure is not limited thereto.

The present disclosure comprises the method of producing Raman-active nanoparticles described above.

Hereinafter, the production method according to the present disclosure will be described in detail. In this case, a metal core, a Raman reporter, a self-assembled monolayer, a metal shell, an analyte, a receptor, and the like are similar or identical to those described above for the Raman-active particle. Therefore, the method of producing Raman-active particles according to the present disclosure comprises all of the details described above for the Raman-active nanoparticle.

The method of producing Raman-active nanoparticles according to the present disclosure is a method of producing Raman-active nanoparticles for surface-enhanced Raman spectroscopy (SERS), the method comprising: a) forming a first self-assembled monolayer comprising a Raman reporter satisfying the following Chemical Formula 1 on a spherical plasmonic metal core; and b) forming a plasmonic metal shell that surrounds the metal core on which the self-assembled monolayer is formed and has surface irregularities using a reaction solution in which a buffer solution, the metal core on which the self-assembled monolayer is formed, and a plasmonic metal precursor are mixed:

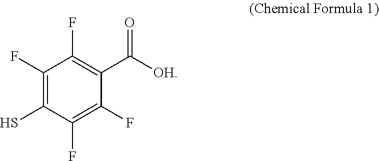 [SMILES: O=C(O)c1c(F)c(F)c(S)c(F)c1F]

In the Raman-active nanoparticle of the present disclosure, a Raman reporter satisfying Chemical Formula 1 is used to form a self-assembled monolayer and then a plasmonic metal shell having surface irregularities is formed, and therefore, the surface irregularities may have a more uniform size than the Raman-active particles according to the related art, and the surface irregularities may be uniformly formed in all directions based on the center of the metal core. Accordingly, the size and shape of the Raman-active nanoparticle of the present disclosure are uniform, which is advantageous for Raman signal enhancement. As a result, the Raman-active nanoparticle of the present disclosure may have improved Raman signal intensity compared to the Raman-active particles according to the related art.

In addition, since the Raman-active nanoparticle of the present disclosure uses the Raman reporter satisfying Chemical Formula 1, Raman-active nanoparticles having a core-shell structure having a large nanogap thickness and surface irregularities formed with a uniform size compared to the Raman-active particles may be produced with excellent reproducibility compared to the Raman-active particles according to the related art.

Furthermore, in general, in producing the Raman-active particles, an organic surfactant is used in a well-known manner to suppress growth of a metal to be nanoparticulated, induce the growth to a specific direction, and/or stabilize the nanoparticle while providing appropriate reducibility for metal nanoparticulation and designed shaping, and an organic acid or an organic acid that may substitute a surfactant is also used.

However, since an organic surfactant (or an organic surfactant and an organic substance derived from an organic acid) that is harmful to a living body and may affect a biochemical material binds to the metal nanoparticle synthesized by such a method, a post-treatment process, such as capping the particle with a capping material having biocompatibility or substituting a harmful surface functional group of the organic surfactant or the like with another functional group having biocompatibility, is essentially required, in order to be used in the biofield. Since the capped metal nanoparticle has a reduced SERS effect by the capping material, when the particles are used in biosensing or bioimaging, high sensitivity may be limited and detection reliability may also be reduced, and when the organic surfactant is to be substituted with a biocompatible functional group, it is difficult to completely substitute the organic surfactant that substantially binds to a metal material with a significantly strong binding force with a biocompatible functional group; thus, toxicity still remains in the metal nanoparticle.

On the other hand, in the method of producing Raman-active nanoparticles according to another exemplary embodiment of the present disclosure, a self-assembled monolayer comprising a Raman reporter is formed on a metal core having a bare metal surface, then, a buffer solution that already has biocompatibility and a solution containing a metal precursor are used to form a metal shell, and the produced Raman-active nanoparticles are free from the organic surfactant that is harmful to a living body; thus, the Raman-active nanoparticles of the present disclosure have biocompatibility without an additional post-treatment process. Accordingly, in the method of producing Raman-active nanoparticles according to an exemplary embodiment, the reaction solution may not contain a surfactant (organic surfactant), and further, the reaction solution may not contain both a surfactant and an organic acid.

In addition, in the method of producing Raman-active nanoparticles according to the present disclosure, as Raman-active nanoparticles are produced by a simple process of attaching the Raman reporter to the metal core and using a solution containing the buffer solution and the metal precursor to form a metal shell, the Raman-active particles may be mass-produced in a short time at low cost, and thus, commerciality may be excellent.

Therefore, the method of producing Raman-active nanoparticles according to the present disclosure may be free from the problem of a reduced SERS effect by capping to have excellent reproducibility and reliability, may have sensitivity capable of implementing single molecule detection, and may mass-produce the Raman-active nanoparticles having biocompatibility at low cost by a simple process without an additional post-treatment.

The method of producing Raman-active nanoparticles of the present disclosure may further comprise, after step b), c) forming a second self-assembled monolayer comprising a Raman reporter satisfying Chemical Formula 1 on the plasmonic metal shell.

In this case, the second self-assembled monolayer is formed by binding the Raman reporter to the metal shell, such that further improved sensitivity may be obtained.

As a specific example, step a) of forming the first self-assembled monolayer comprising the Raman reporter satisfying Chemical Formula 1 on the metal core may comprise preparing a mixed solution containing a metal core and the Raman reporter and performing ultrasonic stirring on the mixed solution.

Specifically, step a) may comprise a1) mixing a metal core and the Raman reporter so that molar concentrations thereof are 0.01 to 1 nM and 10 to 1,000 μm, respectively, to prepare a mixed solution; a2) performing a reaction at room temperature for 10 to 30 minutes while performing ultrasonic stirring on the mixed solution; and a3) separating and recovering the metal core to which the Raman reporter is fixed. In this case, the mixed solution may be an aqueous mixed solution, but is not limited thereto.

In the step of forming the first self-assembled monolayer comprising the Raman reporter on the metal core, the ultrasonic stirring is performed for the time in the above range, such that formation of the Raman reporter as a double layer is suppressed, and thus, the nanogap corresponding to the thickness of the self-assembled monolayer described above may be formed.

In a specific example, the nanogap corresponding to the thickness of the self-assembled monolayer may be formed by the Raman reporter satisfying the following Chemical Formula 1:

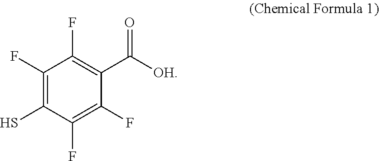 [SMILES: O=C(O)c1c(F)c(F)c(S)c(F)c1F]

Specifically, the first self-assembled monolayer formed by the Raman reporter satisfying Chemical Formula 1 may have a spherical shape that is significantly similar to the radius of curvature of the spherical metal core, and the size of the nanogap corresponding to the thickness of the self-assembled monolayer is uniformly formed; thus, an improved Raman signal intensity may be obtained. In particular, due to such an effect, when an average diameter of the plasmonic metal core is 20 to 100 nm, specifically 30 to 80 nm, and more specifically 40 to 60 nm, the self-assembled monolayer formed by an interaction with the Raman reporter satisfying Chemical Formula 1 forms a SERS hot spot area that is uniformly present in the entire area of the Raman-active nanoparticle, such that Raman signal intensity may be significantly improved.

After performing step a), b) forming a metal shell that surrounds the metal core to which the Raman reporter is fixed using a reaction solution in which a buffer solution, the metal core on which the self-assembled monolayer is formed, and a metal precursor are mixed may be performed.

In step b) of forming the metal shell, a hydrogen ion concentration index (pH) of the buffer solution comprised in the reaction solution may be 5.5 to 7.5, and preferably 6 to 7.

A mole ratio between a buffer in the buffer solution and the metal precursor (a mole ratio obtained by dividing the number of moles of the buffer by the number of moles of the metal precursor) may be 10 to 100, and preferably 20 to 80.

The thickness and the shape of the metal shell formed to surround the metal core may be changed depending on the pH environment of the buffer solution and the concentration of the reaction material, and as described above, in order to form a thin metal shell that completely surrounds the Raman reporter fixed to the metal core and form a metal shell having surface irregularities having a uniform size by the metal fine particles, it is preferable that the pH of the buffer solution and the mole ratio between the buffer and the metal precursor are in the ranges described above.

As a specific example, the buffer solution may comprise one or more selected from 4-(2-hydroxyethyl)-1-piperazineethanesulfonic acid (HEPES), 4-(2-hydroxyethyl) piperazine-1-ethanesulfonic acid (MES), phosphated buffered saline (PBS), tris(2-amino-2-hydroxymethyl-propane-1,3-idol), phosphate buffer (PB), 3-(N-morpholino) propanesulfonic acid (MOPS), 3-[[1,3-dihydroxy-2-(hydroxymethyl) propan-2-yl]amino]propane-1-sulfonic acid (TAPS), and piperazine-N,N′-bis(2-ethanesulfonic acid) (PIPES). The buffer in the buffer solution may serve as a weak reducing agent that reduces a metal, and a surfactant for stabilizing the Raman-active particles to be produced by the buffer in the buffer solution may be excluded.

The metal of the metal precursor may be gold, silver, platinum, palladium, nickel, aluminum, copper, a mixture thereof, an alloy thereof, or the like. However, the metal of the metal precursor is preferably gold or silver independently of the metal of the metal core in consideration of biosafety. As an example, the metal of the metal precursor may be the same metal as the metal of the metal core.

The metal precursor according to an advantageous example may be a gold precursor such as HAuCl₄, HAuBr₄, NaAuCl₄, AuCl₃·3H₂O, NaAuCl₄·2H₂O, or a mixture thereof, or a silver precursor such as AgNO₃, but is not limited thereto.

In a specific example, in step b), the buffer solution, the metal precursor solution, and the dispersion of the metal core to which the Raman reporter is fixed may be mixed to prepare a reaction solution, and the reaction may be performed at a temperature of 15 to 40° C., specifically at a temperature of 15 to 35° C., more specifically at a temperature of 15 to 25° C., and still more specifically at room temperature of 21 to 25° C. The metal shell may be produced by reacting the reaction solution for 10 minutes to 50 minutes, and specifically 20 minutes to 40 minutes, but the present disclosure is not limited to the reaction time of the reaction solution.

In this case, stirring may be performed at the time of the reaction, the stirring may be performed at a speed of 500 to 1,200 rpm, specifically 600 to 1,000 rpm, and more specifically 600 to 800 rpm, and the reaction may be completed by adding an excessive amount of deionized water to the reaction solution to suppress the synthesis reaction.

A molar concentration of the buffer in the buffer solution may be 10 to 200 mM, and specifically 10 to 100 mM, and a molar concentration of the metal precursor in the metal precursor solution may be 1 to 20 mM, and specifically 1 to 10 mM. A molar concentration of the metal core in the dispersion of the metal core to which the Raman reporter is fixed may be 0.01 to 1.0 nM, and specifically 0.01 to 0.5 nM, but is not limited thereto.

The buffer solution and the metal precursor solution may be mixed so that the mole ratio between the buffer and the metal precursor described above is satisfied, and the metal core dispersion may be mixed so that the mole ratio of the metal precursor to the metal core is 1:1×10⁻⁷ to 1×10⁻⁵. In this case, the metal precursor solution and the metal core dispersion may be first mixed and then the buffer solution may be mixed so that the metal shell may be uniformly formed on the metal core(s).

Specifically, step b) may comprise b1) mixing a metal precursor solution and a metal core dispersion to prepare a precursor-metal core mixed solution; b2) mixing a buffer solution with the precursor-metal core mixed solution to prepare a reaction solution and reacting the reaction solution at a temperature of 15 to 40° C., and advantageously at room temperature, to produce Raman-active nanoparticles; and b3) separating and recovering the produced Raman-active particles, adding the recovered Raman-active nanoparticles to a buffer solution (an additional buffer solution), and storing the solution at a temperature of 1 to 10° C., and specifically at a temperature of 1 to 5° C.

By step b), the Raman-active nanoparticles comprising the metal core, the self-assembled monolayer of the Raman reporter surrounding the metal core, and the metal shell surrounding the self-assembled monolayer may be produced.

In a specific example, the method of producing Raman-active nanoparticles may further comprise, after step b), d) fixing a receptor that binds (specifically binds) to an analyte to the metal shell. Step d) may be performed by mixing the receptor with the prepared Raman-active nanoparticle dispersion, and fixing may be performed depending on a protocol known for each receptor.

After performing step b), c) forming a second self-assembled monolayer comprising a Raman reporter satisfying Chemical Formula 1 on the plasmonic metal shell may be performed:

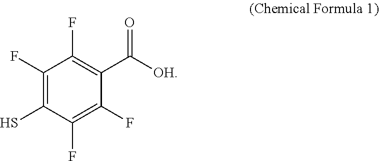 [SMILES: O=C(O)c1c(F)c(F)c(S)c(F)c1F]

Specifically, the second self-assembled monolayer comprising the Raman reporter satisfying Chemical Formula 1 may have a spherical shape that is significantly similar to the radius of curvature of the spherical metal shell, and may improve sensitivity.

In a specific example, the method of producing Raman-active nanoparticles may further comprise, after step c), d) fixing a receptor that binds (specifically binds) to an analyte to the metal shell. Step d) may be performed by mixing the receptor with the prepared Raman-active nanoparticle dispersion, and fixing may be performed depending on a protocol known for each receptor.

In addition, before step a), washing the metal core using an organic solvent or the like may be further performed so that the spherical metal core has a bare metal surface, and the washing is only performed when needed.

Hereinafter, examples of the present disclosure will be described in detail so that the present disclosure may easily be implemented by those skilled in the art to which the present disclosure pertains. However, the present disclosure may be implemented in various different forms and is not limited to examples described herein.

### [Example 1] Production of Raman-Active Nanoparticles 1

4 mL of a solution of spherical Au colloid having a diameter of 50 nm (EM.GC50, BBI solution) was centrifuged at 4,000 rpm for 10 minutes to remove a supernatant, and then was mixed with 4 mL of a 0.1 mM bis(p-sulfonatophenyl)phenylphosphine dihydrate dipotassium salt (BSPP) solution, thereby preparing an Au core dispersion having a molar concentration of 0.1 nM.

4 mL of the Au core dispersion and 16 μL of a 10 mM 2,7-mercapto-4-methylcoumarin (MMC) solution were mixed (the final concentration of MMC was 40 μM), and the mixed solution was sonicated for 10 minutes and then centrifuged at 4,000 rpm for 15 minutes to recover a dispersion of an Au core in which a first self-assembled monolayer of MMC, which is a Raman reporter, was formed.

To 1 mL of the recovered dispersion of the Au core in which the first self-assembled monolayer of MMC was formed, 5 mL of a 50 mM HEPES buffer solution at pH 6.5 and 1 mL of 5 mM HAuCl₄ (254169, Sigma-Aldrich) were added to prepare a reaction solution, and the reaction solution was stirred at room temperature and 600 rpm for 30 minutes. At this time, the pH of the buffer solution was adjusted using 1 mM NaOH and 1 mM HCl.

Thereafter, the reaction solution was sequentially centrifuged at 3,000 rpm for 10 minutes to remove a supernatant, 4 mL of 0.1 mM BSPP was mixed with the reaction solution from which the supernatant was primarily removed, and then centrifugation was performed at 3,000 rpm for 10 minutes to secondarily remove a supernatant.

Thereafter, 4 mL of 0.1 mM BSPP was additionally mixed with the recovered reaction solution, centrifugation was performed at 3,000 rpm for 10 minutes to remove a supernatant, and then, 4 mL of 0.1 mM BSPP was added to terminate the reaction.

### [Example 2] Production of Raman-Active Nanoparticles 2

16 μL of a 10 mM 2,7-mercapto-4-methylcoumarin (MMC) solution was mixed with the Raman-active nanoparticles finally obtained in Example 1, and then the mixed solution was sonicated for 10 minutes and then centrifuged at 3,000 rpm for 10 minutes to recover a reaction solution of an Au core-shell in which a second self-assembled monolayer of MMC, which is a Raman reporter, was formed.

Thereafter, the reaction solution was sequentially centrifuged at 3,000 rpm for 10 minutes to remove a supernatant, 4 mL of 0.1 mM BSPP was mixed with the reaction solution from which the supernatant was primarily removed, and then centrifugation was performed at 3,000 rpm for 10 minutes to secondarily remove a supernatant. Thereafter, 4 mL of 0.1 mM BSPP was additionally mixed with the recovered reaction solution, centrifugation was performed at 3,000 rpm for 10 minutes to remove a supernatant, and then, 4 mL of 0.1 mM BSPP was added to terminate the reaction.

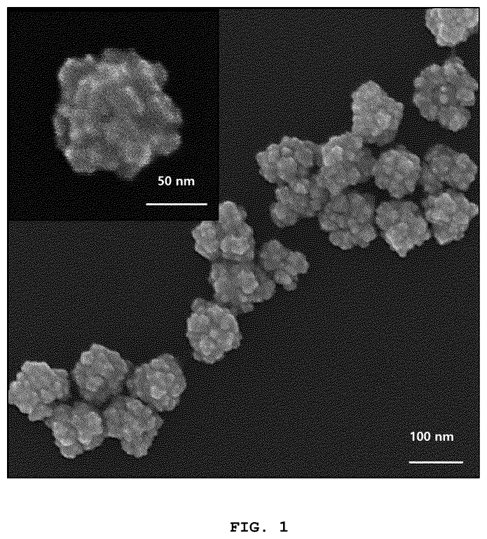

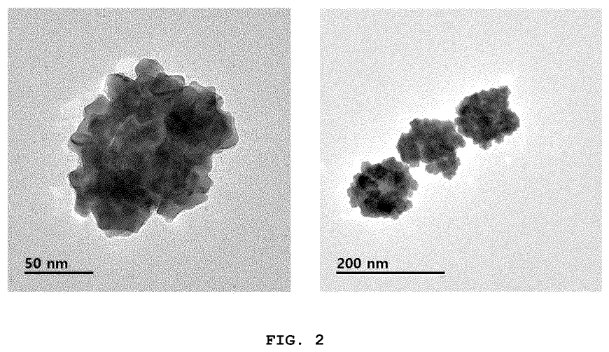

FIG. 1 is a scanning electron microscope (SEM) image obtained by observing the Raman-active nanoparticles of Example 1, and FIGS. 2A and 2B are transmission electron microscope (TEM) images.

It was confirmed that the self-assembled monolayer of the Raman reporter was positioned between the Au core and the polycrystalline Au shell formed of Au fine particles, and a uniform nanogap having a thickness of 1.0 nm was formed in the entire particle area. The average size of the Raman-active nanoparticles of Example 1 was 121 nm, and the average size of the Raman-active nanoparticles of Example 2 was 123 nm.

In addition, it was seen that the surface irregularities were uniformly formed on the shell due to the protrusion of the Au fine particles, and it was confirmed that the surface irregularities due to the protrusion of the fine particles were uniformly formed in all directions based on the center of the core particle.

In the examples, uniform SERS activity based on the particle was exhibited because isotropic Raman activity in the nanoparticle was exhibited by bumpy irregularities uniformly formed in the entire area of the surface of the metal shell, and since each Raman-active nanoparticle had a uniform size, there is almost no deviation in Raman activity between the particles, and uniform SERS activity between the particles was exhibited.

In Example 1, it was confirmed that a SERS signal was detected in 85% of all the Raman-active nanoparticles, and in Example 2, it was confirmed that a SERS signal was detected in 90% of all the Raman-active nanoparticles.

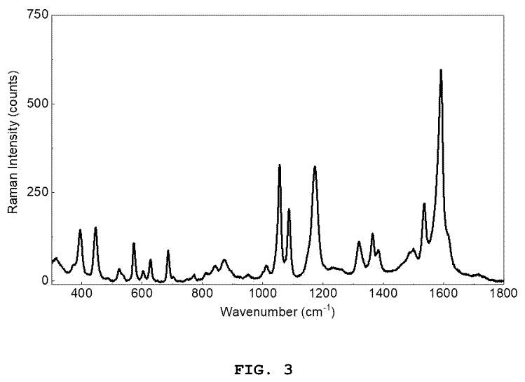

FIG. 3 is a diagram showing a spectrum of light emitted from a subject by excitation light using Example 1. In the graph, the horizontal axis represents the reciprocal of the wavelength of the emitted light, that is, the wavenumber, and the vertical axis represents the intensity of the emitted light. Analysis was performed by irradiating the subject with a 785 nm light source. Referring to FIGS. 2A and 2B, it can be seen that Raman peaks appear with high intensity at 1,055.656 cm⁻¹, 1,172.811 cm⁻¹, and 1,590.806 cm⁻¹.

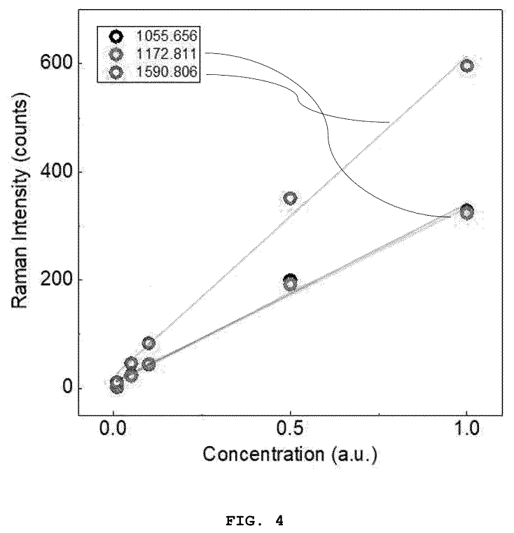

In addition, FIG. 4 is a diagram showing the result of evaluating Raman signal intensity using a slope value obtained by linear regression analysis for all concentration values. The coefficient of determination was at a level of 0.993, and high reliability of the linear regression analysis method was confirmed.

From this, it was seen that Examples 1 and 2 were advantageous in detecting the target material because the Raman signal intensity was improved.

This was because in the Raman-active nanoparticles of the examples described above, the nanogap and surface irregularities having uniform sizes were uniformly formed in all directions based on the center of the metal core particle, and the size and shape of each Raman-active nanoparticle were also uniform.

As set forth above, the Raman-active nanoparticle according to an exemplary embodiment of the present disclosure has a core-shell structure comprising a spherical plasmon active core and a plasmon active shell having surface irregularities by fine particles, and comprises a first self-assembled monolayer that is positioned between the core and the shell and comprises a Raman reporter satisfying Chemical Formula 1, such that the Raman-active nanoparticle may have hot spots that are uniformly present in the entire area of the nanoparticle, and the sensitivity may be significantly improved without inhibiting the isotropic Raman activity in the nanoparticle and the uniformity of the Raman activity between the nanoparticles by the bumpy irregularities formed uniformly in the entire area of the surface of the metal shell.

In addition, the Raman-active nanoparticle according to an exemplary embodiment of the present disclosure has uniform Raman activity based on the nanoparticle, has the advantage of implementing detection with reproducibility as a Raman signal is detected in 80% or more of the total number of Raman-active nanoparticles, and has excellent detection ability at a single molecule level.

In addition, the Raman-active nanoparticle according to an exemplary embodiment of the present disclosure has excellent biocompatibility, which is suitable for detecting a specific biomarker or a cell surface receptor, and comprises a self-assembled monolayer having improved chemical stability, which is excellent in detection reliability.

In addition, in the Raman-active nanoparticle according to an exemplary embodiment of the present disclosure, well-defined hot spots are continuously present in the entire area of the nanoparticle, and the Raman reporter is uniformly positioned in the well-defined hot spots, such that a biochemical material (biomaterial) having a several to several tens of micrometers may also be reproducibly detected.

In addition, the method of producing Raman-active nanoparticles according to another exemplary embodiment of the present disclosure is an extremely simple method of mixing a buffer solution, a metal precursor, and a spherical metal core at room temperature without aid of a surfactant, and has excellent commerciality because Raman-active nanoparticles having the advantages described above may be mass-produced in a short time.

Hereinabove, although the present disclosure has been described by specific matters, limited exemplary embodiments, and drawings, they have been provided only for assisting in the entire understanding of the present disclosure. Therefore, the present disclosure is not limited to the exemplary embodiments. Various modifications and changes may be made by those skilled in the art to which the present disclosure pertains from this description.

Therefore, the spirit of the present disclosure should not be limited to the described exemplary embodiments, but the claims and all modifications equal or equivalent to the claims are intended to fall within the spirit of the present disclosure.

1. A Raman-active nanoparticle comprising:
a spherical plasmonic metal core;
a plasmonic metal shell having surface irregularities; and
a first self-assembled monolayer that binds to each of the core and the shell, is positioned between the core and the shell, and comprises a Raman reporter satisfying the following Chemical Formula 1:

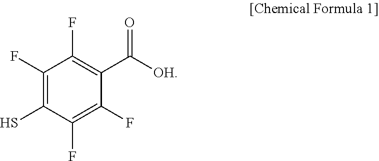 [SMILES: O=C(O)c1c(F)c(F)c(S)c(F)c1F]

2. The Raman-active nanoparticle of claim 1, wherein the surface of the shell comprises a second self-assembled monolayer comprising a Raman reporter satisfying Chemical Formula 1.

3. The Raman-active nanoparticle of claim 1, wherein the Raman-active nanoparticle has a strong Raman signal at 1,035 to 1,075 cm⁻¹, 1,150 to 1,190 cm⁻¹, and 1,570 to 1,610 cm⁻¹ when irradiated with a 785 nm light source.

4. The Raman-active nanoparticle of claim 1, wherein the plasmonic metal shell comprises plasmonic metal fine particles having an average size of 0.3 D to 1 D based on a diameter (D) of the metal core, and has surface irregularities due to the plasmonic metal fine particles.

5. The Raman-active nanoparticle of claim 4, wherein in the plasmonic metal shell, an inner shape of the shell in contact with the self-assembled monolayer is a spherical shape.

6. The Raman-active nanoparticle of claim 4, wherein an average diameter of the plasmonic metal core is 20 to 100 nm.

7. The Raman-active nanoparticle of claim 1, wherein a thickness of the self-assembled monolayer is 0.5 to 2.0 nm.

8. The Raman-active nanoparticle of claim 1, wherein the plasmonic metal core and the plasmonic metal shell are independently one or more metals selected from gold, silver, platinum, palladium, nickel, aluminum, and copper.

9. The Raman-active nanoparticle of claim 8, wherein the plasmonic metal core and the plasmonic metal shell are the same metal.

10. The Raman-active nanoparticle of claim 1, further comprising a receptor that is fixed to the plasmonic metal shell and binds to an analyte.

11. The Raman-active nanoparticle of claim 1, wherein a surface-enhanced Raman scattering signal in Raman mapping is detected in 80% or more of the total number of Raman-active nanoparticles.

12. The Raman-active nanoparticle of claim 1, wherein the Raman-active nanoparticle is used for near-infrared excitation light having a wavelength of 780 to 790 nm.

13. A method of producing Raman-active nanoparticles, the method comprising:
a) forming a first self-assembled monolayer comprising a Raman reporter satisfying the following Chemical Formula 1 on a spherical plasmonic metal core; and
b) forming a plasmonic metal shell that surrounds the metal core on which the self-assembled monolayer is formed and has surface irregularities using a reaction solution in which a buffer solution, the metal core on which the self-assembled monolayer is formed, and a plasmonic metal precursor are mixed:

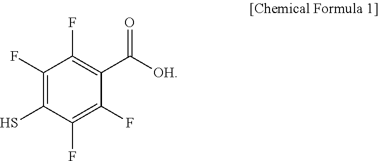 [SMILES: O=C(O)c1c(F)c(F)c(S)c(F)c1F]

14. The method of claim 13, further comprising, after step b), c) forming a second self-assembled monolayer comprising a Raman reporter satisfying Chemical Formula 1 on the plasmonic metal shell.

15. The method of claim 13, wherein a mole ratio obtained by dividing the number of moles of a buffer in the buffer solution by the number of moles of the plasmonic metal precursor is 10 to 100.

16. The method of claim 13, wherein a molar concentration of a buffer in the buffer solution is 10 to 200 mM.

17. The method of claim 13, wherein a diameter of the plasmonic metal core is 20 to 100 nm.

18. The method of claim 13, further comprising, after step b), d) fixing a receptor that binds to an analyte to the plasmonic metal shell.

19. The method of claim 14, further comprising, after step c), d) fixing a receptor that binds to an analyte to the plasmonic metal shell.
